(19) United States
(12) Patent Application Publication
(10) Pub. No.: US 2008/0255380 A1
(43) Pub. Date: Oct. 16, 2008

(54) Process and Intermediates for the Preparation of Sulfonyl Derivatives of Cholecalciferol

(75) Inventors: Michal Chodynski, Pruszkow (PL); Malgorzata Krupa, Warszawa (PL); Hanna Fitak, Warszawa (PL); Jerzy Winiarski, Warszawa (PL); Teresa Ryznar, Warszawa (PL); Bartlomiej Gorecki, Warszawa (PL); Wieslaw Szelejewski, Warszawa (PL); Andrzej Kutner, Warszawa (PL)

Correspondence Address: MATTHIAS SCHOLL, 14781 MEMORIAL DRIVE, SUITE 1319, HOUSTON, TX, 77079, US

(73) Assignee: INSTYTUT FARMACEUTYCZNY, WARSZAWA (PL)

(21) Appl. No.: 11/568,977

(22) PCT Filed: May 13, 2005
(86) PCT No.: PCT/PL2005/000030

§ 371 Date: Mar. 4, 2008

(30) Foreign Application Priority Data
May 14, 2004 (PL) P-368012

(51) Int. Cl. C07C 401/00

(52) U.S. Cl. 552/653

(57) ABSTRACT

Preparation of sulfonyl derivatives of cholecalciferol of Formula 1, wherein R₁ is a protective group, preferably a t-butyl(dimethyl)silyl, and R₂ is a heterocyclic group, such as a 2-thiazolyl, a 2-benzothiazolyl, a 1-phenyl-1H-tetrazo-5-yl, a 2-pyridyl, a 2-pyrimidynyl, a 1-isochinolinyl, a 1-methyl-2-imidazyl, or a 4-alkyl-1,2,4-triazo-3-yl, comprises the conversion of the hydroxyl derivative of cholecalciferol into the corresponding sulfide followed by its oxidation to the respective sulfone characterized by the use of a hydroxyl derivative of cholecalciferol as a starting material, in which the triene system is protected as a Diels-Alder adduct, and in particular as an adduct with sulfur dioxide of the Formula 2a. Novel are also the derivatives of Formula 3a and 4a, isolated in the process provided by the invention.

### CROSS-REFERENCE TO RELATED APPLICATIONS

This is a National Stage Application of International Patent Application No. PCT/PL 2005/000030, with an international filing date of May 13, 2005, which is based on a Polish Patent Application No. P-368012, filed May 14, 2004. The contents of both of these specifications are incorporated herein by reference.

### BACKGROUND OF THE INVENTION

1. Field of the Invention

This invention relates to a process for the preparation of sulfonyl derivatives of cholecalciferol and to novel intermediate compounds isolated in the embodiments of this process. Especially, this invention relates to a process for the preparation of cholecalciferol derivatives with a sulfonyl substituent at C-22, which are useful for the synthesis of aliphatic side chain-modified analogues of Vitamin D. In particular, C-22 sulfonyl derivatives of cholecalciferol may be used in the synthesis of pharmacologically-active vitamin D analogs having multiple bonds at C-22, preferably a double bond, such as calcipotriol.

2. Description of the Prior Art

As described in the International Patent Publication No. WO 03/087048, the preparation of cholecalciferol derivatives of Formula 1 having a C-22 sulfonyl substituent comprises oxidation of the Vitamin D sulfide derivative of Formula 3 obtained from the hydroxyl derivative of Formula 2, wherein with reference to the accompanying Scheme I in FIG. 1, R₁ is a protective group, R₂ is a heterocyclic group, such as a 2-thiazolyl, a 2-benzothiazolyl, a 1-phenyl-1H-tetraz-5-yl, a 2-pyridyl, a 2-pyrimidynyl, a 1-isochinolinyl, a 1-methyl-2-imidazyl, or a 4-alkyl-1,2,4-triaz-3-yl.

Due to the presence of a triene system which is sensitive to oxidizing agents, there is a necessity to carry out the oxidation at moderate temperatures. At such conditions, however, significant amounts of intermediate S-oxides are formed in addition to the main product—the sulfonyl of Formula 1. The possibility of raising the temperature of the reaction in the described process so as to completely transform the intermediate sulfides to the corresponding sulfonyl compounds is very limited, due to a competitive oxidation reaction of the triene system. A complete separation of the final sulfonyl derivative from the intermediate sulfides is associated with significant preparational difficulties, particularly at scale-up, and a decrease of the reaction yield.

Thus, a protection of the triene system was considered appropriate, which would not only allow for effective oxidation of sulfide to sulfone, but would also not result in excessive prolongation of the synthetic procedure related to an additional step of deprotection, and moreover would allow for a reconstruction of a desired double bond system in the oxidation product.

Protection methods for triene systems are known in the art and are often applied in vitamin D chemistry.

For example, as a result of solvolysis of vitamin D tosylate in aqueous acetone, (6R)-hydroxy-3,5-cyclo-vitamin D system comprising only isolated double bonds can be obtained (Mazur and Sheves, J. Am. Chem. Soc. 97, 6249, 1975). This system was proved particularly convenient for the functionalization of ring A of vitamin D at the biologically significant C-1 carbon. Reconstruction of the triene system was carried out in acidic conditions, leading to a mixture of (5E,7E) and (5Z,7E) isomers. Barton et al. (J. Chem. Soc. Perkin Trans 1, 829, 1976) obtained an isomeric mixture of tricarbonyl iron complexes coordinating double bonds 5, 6 and 10, 19 of the triene system. The reconstruction of the triene system was carried out under the mild conditions with ferric chloride. However, none of these systems were proper for the oxidating and regioselective double bond cleavage in the side chain.

Not until Aberhart (J. Org. Chem., 41, 2098, 1976) and, independently, Reischl (Monatsh. Chem., 113, 439, 1982), was it observed that the vitamin D triene system forms Diels-Alder adducts with dienophiles, such as the 4-phenyl-1,2,4-triazoline-3,5-dione or the phthaloyl-1,4-dione. In the reaction of a mixture of isomeric C-6 adducts with ozone, a regioselective double bond cleavage in the side chain is observed. However, the deprotection of the (5E,7E)-Vitamin D in alkaline conditions (e.g. potassium hydroxide in boiling methanol, butanol or ethylene glycol) gives only a limited yield and only after many hours of reaction time (M. Chodyński et al., Steroids, 67, 789, 2002).

Particularly convenient was the protection of the triene system in the adduct form with sulfur dioxide, developed independently by Yamada (Chem. Lett., 583, 1979) and Reischl (Helv. Chim. Acta, 62, 1763, 1979). These adducts are obtained as a mixture of epimers at the C-6 carbon in a quantitative yield, by passing sulfur dioxide through a benzene-water solution of vitamin D. Thermolysis of the adduct mixture in boiling ethanol results in a reconstruction of the triene isomer (5E,7E).

### BRIEF DESCRIPTION OF THE DRAWINGS

The objects and advantages of the present invention will become more readily apparent after reading the ensuing description of the non-limiting illustrative embodiment and viewing the accompanying drawings, in which

FIG. 1 shows Scheme I illustrating a prior art method for the preparation of cholecalciferol derivatives having a C-22 sulfonyl substituent;

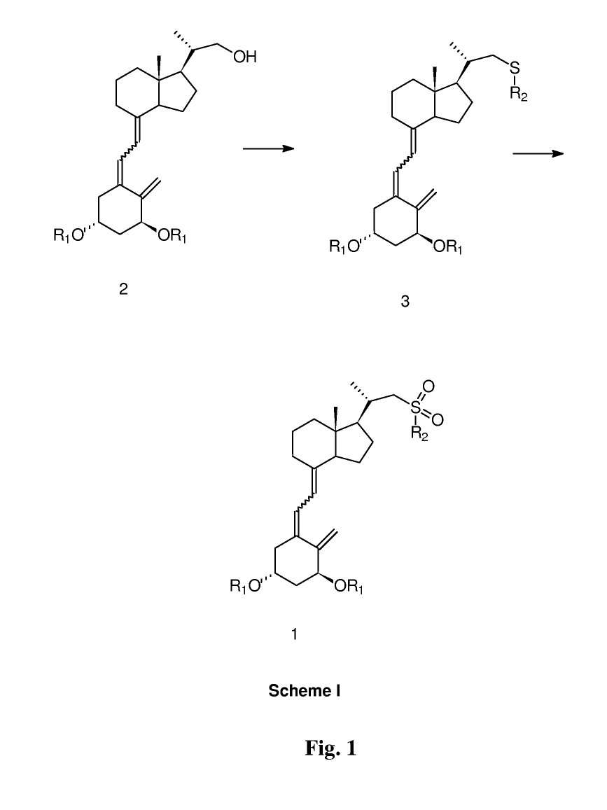

FIG. 2 shows Scheme II illustrating a process for the preparation of cholecalciferol derivatives having a C-22 sulfonyl substituent according to the invention; and

FIG. 3 shows Scheme III illustrating the synthesis of sulfonyl derivatives of cholecalciferol and novel intermediates leading thereto according to one embodiment of the invention.

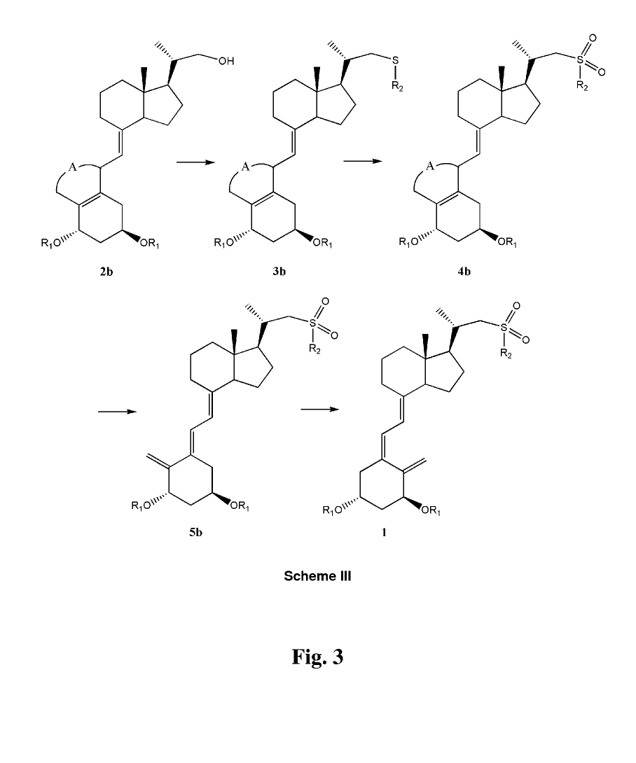

### DETAILED DESCRIPTION OF THE INVENTION

It has recently been observed that it is beneficial to use Diels-Alder-protected adducts of C-22 sulfide-derivatives of cholecalciferol in the oxidation of sulfide to sulfone even though this involves the necessity of inversion of configuration of the thus obtained sulfone and results in an extension of the synthesis by an additional photoisomerization step.

The technological advantages of this route include the possibility of applying higher temperatures in the oxidation of sulfide to sulfone, a higher yield of the process resulting from a nearly-complete conversion of the substrate, and absence of the S-oxide byproducts.

Moreover, methods taught herein provide for a sensitized photoisomerization of compounds having both the (5E,7E) triene system and the C-22 sulfonyl moiety, which photoisomerization was difficult to predict theoretically given the prior art literature on this topic. Specifically, during the photoisomerization process, regrouping or decomposition of the desired arrangement was not observed, and the process was carried out with high a yield that exceeded 80%.

With reference to Scheme III, the process for the preparation of sulfonyl derivatives of cholecalciferol of Formula 1 according to the invention described herein comprises the use of a hydroxyl derivative of cholecalciferol of Formula 2b as a starting material, in which the triene system of the hydroxyl derivative is protected as an Diels-Alder adduct; this adduct is then subjected to the reaction with a thiol of formula HS—R₂; then the resultant adduct of a sulfide of Formula 3b is oxidized to an adduct of sulfone of Formula 4b; in the following step, the protective moiety is removed by thermolysis; and the resulting (5E,7E) sulfone of Formula 5b is subjected to a sensitized photoisomerization to give the (5Z,7E) sulfone of Formula 1; wherein R₁ is a protective group, preferably a t-butyl(dimethyl)silyl, R₂ is a heterocyclic group, such as a 2-thiazolyl, a 2-benzothiazolyl, a 1-phenyl-1H-tetrazo-5-yl, a 2-pyridyl, a 2-pyrimidynyl, a 1-isochinolinyl, a 1-methyl-2-imidazyl, or a 4-alkyl-1,2,4-triazo-3-yl, and A independently and at each occurrence represents a dienophile moiety derived from a dienophile having undergone a Diels-Alder reaction and selected from the group consisting of sulfur dioxide, 4-phenyl-1,2,4-triazoline-3,5-dione and phthaloyl-1,4-dione. For example, if the dienophile is SO₂, A is —S(O₂)—.

The Diels-Alder adduct 2b may be any adduct formed in the reaction of the starting hydroxyl derivative of cholecalciferol of Formula 2 with a dienophile. The dienophile is any suitable dienophile that participates in a Diels-Alder reaction with a compound of Formula 2, including without limitation sulfur dioxide, 4-phenyl-1,2,4-triazoline-3,5-dione or phthaloyl-1,4-dione.

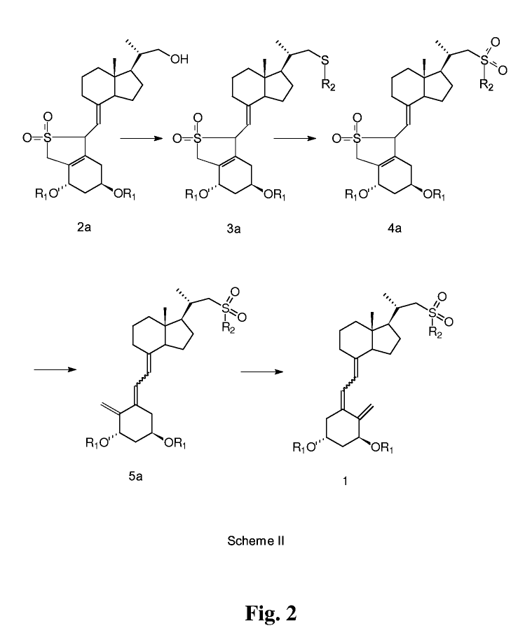

In the preferred embodiment of the invention, the starting hydroxyl derivative of cholecalciferol is protected as an adduct with sulfur dioxide of Formula 2a. When the adduct of sulfur dioxide is used, the triene is not only easily protected, but it is as easily deprotected after the oxidation step. This embodiment is presented in FIG. 2., Scheme II.

Sulfide adducts of Formula 3a and sulfone adducts of Formula 4a isolated in the method provided by the invention are novel cholecalciferol derivatives, not heretofore described in the literature, wherein R₁ is a protective group, and R₂ is a heterocyclic group, such as a 2-thiazolyl, a 2-benzothiazolyl, a 1-phenyl-1H-tetrazo-5-yl, a 2-pyridyl, a 2-pyrimidynyl, a 1-isochinolinyl, a 1-methyl-2-imidazyl, or a 4-alkyl-1,2,4-triazo-3-yl. Particularly valuable are novel derivatives of Formula 3a and 4a according to the invention, wherein R₁ is t-butyl(dimethyl)silyl, and R₂ is 2-benzothiazolyl.

According to the invention, an adduct of the hydroxyl derivative of cholecalciferol of Formula 2a, wherein R₂ is a heterocyclic group, is converted directly into an adduct of the sulfide of Formula 3a under Mitsunobu reaction conditions with a thiol of formula HS—R₂, preferably using 2-mercaptobenzothiazole and triphenylphosphine in the presence of diisopropyl azodicarboxylate in methylene chloride at lower temperatures. Oxidation to the sulfone of Formula 4a is carried out with ammonium molybdate tetrahydrate-hydrogen peroxide system in ethanol under elevated temperatures.

Adduct protection is removed by thermolysis, e.g. with sodium bicarbonate in an alcohol, such as methanol, ethanol, butanol or ethylene glycol, under reflux.

Sulfonyl derivatives of cholecalciferol of Formula 5a comprising the (5E,7E) triene system obtained according to the invention demonstrate a strong tendency to crystallization, which is unique among Vitamin D compounds. This allows for their convenient use in the synthesis of vitamin D analogs to facilitate easy removal of impurities from prior reaction steps, without the necessity of tedious purification of intermediate compounds.

Photoisomerization of the (5E,7E) to the (5Z,7E) triene system is carried out according to the methods known in the field of Vitamin D chemistry, with the yield exceeding 80%. Sulfonyl derivatives of cholecalciferol having the (5Z,7E)-triene system, obtained according to the methods of the invention, are valuable starting materials for the synthesis of various Vitamin D derivatives.

### EXAMPLES

The invention is further illustrated by the following non-limiting examples.

### Example 1

### Preparation of (6RS)-SO₂-(5E,7E)-(1S,3R)-1,3-bis[t-butyl(dimethylsilyl)oxy]-22-thiobenzothiazolyl-23,24-dinor-9,10-secochola-5(10),7-diene adduct [Formula 3a; R₂=thiobenzothiazolyl, R₁=t-butyl(dimethyl)silyl]

In a 1 L round bottom flask with crude (6RS)-SO₂-(5E,7E)-(1S,3R)-1,3-bis[t-butyl(dimethyl-silyl)oxy]-22-hydroxy-23,24-dinor-9,10-secochola-5(10),7-diene adduct (ca. 116 g, content of pure compound ca. 90 g) 500 mL of CH₂Cl₂ was added and the obtained solution was transferred to a dropping funnel. In another round bottom flask (2 L) a suspension of 45 g of 2-thiobenzothiazole in 500 mL of CH₂Cl₂ was prepared. The flask was placed in a cooling bath (0° C.) on a magnetic stirrer. Then 71 g of triphenylphosphine was added in single portion with stirring and the above prepared solution of alcohol of Formula 1 in CH₂Cl₂ was slowly added dropwise. Then, 45 mL of diisopropyl azodicarboxylate was added dropwise. The mixture was vigorously stirred for 90 min at 0° C. The cooling bath was removed; 4 L of brine and 200 mL of water were added. The organic phase was separated, and the residue was extracted with CH₂Cl₂ (2×200 mL). The combined organic phases were dried over anhydrous Na₂SO₄ (80 g). The solution was filtered and concentrated under reduced pressure. The residue was dissolved in toluene-hexane 1:1 (v/v) mixture and loaded onto a chromatography column packed with silica gel (230-400 m, 800 g). The following solvents and their mixtures were used as eluents: hexane 300 ml, hexane-ethyl acetate 2% 1500 mL, hexane-ethyl acetate 4% 1500 mL, hexane-ethyl acetate 6% 1500 mL, hexane-ethyl acetate 8% 1500 mL. The chromatography progress was controlled on TLC using 12% ethyl acetate-hexane as a solvent. Clean fractions were combined and solvents were evaporated under reduced pressure. The residue (ca. 284 g) was dissolved in 600 mL of hexane-toluene 2:1 mixture. The suspension was filtered under reduced pressure using a Buchner funnel. The precipitate remaining on the filter was washed with 1 portion (200 mL) of hexane-toluene 2:1 mixture. The filtrate was concentrated under reduced pressure and thoroughly dried on a vacuum oil pump. Obtained was ca. 160 g of crude product; ¹H-NMR (δ, ppm) 0.06 (12H, m, 2 Si(CH₃)₂), 0.69 (3H, s, 18-CH₃), 0.88 (18H, m, 2 Si—C(CH₃)₃), 1.14 (3H, d, J=6.5 Hz, 21-CH₃), 2.62 and 3.01 (3H, m, 6-H and 22-CH₂), 3.94 (1H, m, 7-H), 4.19 and 4.37 (2H, m, 1-H and 3-H), 4.69 (2H, m, 10′-CH₂), 7.34 and 7.77 (4H, m, Ar—H).

### Example 2

### Preparation of (6RS)-SO₂-(5E,7E)-(1S,3R)-1,3-bis[t-butyl(dimethylsilyl)oxy]-22-sulfonylbenzothiazolyl-23,24-dinor-9,10-secochola-5 (10),7-diene adduct [Formula 4a; R₂=sulfonylbenzothiazolyl, R₁=t-butyl(dimethylsilyl)]

A three-neck flask (4 L) fitted with a mechanical stirrer and a dropping funnel was placed in a water bath and ca. 160 g of crude (6RS)-SO₂-(5E,7E)-(1S,3R)-1,3-bis[t-butyl(dimethylosilyl)oxy]-22-thiobenzothiazolyl-23,24-dinor-9,10-secochola-5(10),7-diene adduct in 400 mL of CH₂Cl₂ and 1200 mL of C₂H₅OH were added. The stirrer was started and a solution of 35 g of ammonium heptamolibdenate hydrate (AHT) in 230 mL of H₂O₂ (35%) was dripped in over ca. 5 minutes. The temperature of the water bath was elevated to 65° C. and the stirring was continued for ca. 2.5 hours until a complete disappearance of the substrate (controlled by TLC). The mixture was cooled to 0° C. and 1400 mL of 10% solution of Na₂SO₃ was dripped in until the disappearance of peroxides (indicator paper). The solvents were removed under reduced pressure, and 1 L of ethyl acetate was added to the residue. The organic phase was separated and the water phase was extracted twice with ethyl acetate (2×500 mL). The combined organic phases were dried over anhydrous Na₂SO₄ (50 g) and filtered into a 3 L flask. The solvents were removed under reduced pressure, and the residue was dried on an oil vacuum pump. Obtained was ca. 145 g of crude mixture of sulfones as a yellow foam oil; IR, v, 2952, 2928, 2883, 2856, 1624, 1472, 1381, 1360, 1323, 1253, 1148, 1121, 1083, 834, 760 cm⁻¹; ¹H-NMR (δ, ppm) 0.06 (12H, m, 2 Si(CH₃)₂), 0.65 (3H, s, 18-CH₃), 0.87 (18H, m, 2 Si—C(CH₃)₃), 1.28 (3H, d, J=6.5 Hz, 21-CH₃), 3.28 and 3.65 (3H, m, 6-H and 22-CH₂), 3.94 (1H, m, 7-H), 4.17 and 4.36 (2H, m, 1-H and 3-H), 4.65 (2H, m, 10′-CH₂), 7.61, 8.02 and 8.22 (4H, m, Ar—H).

### Example 3

### Preparation of (5E,7E)-(1S,3R)-1,3-bis[t-butyl (dimethylosilyl)oxy]-22-sulfonylbenzothiazolyl-23,24-dinor-9,10-secochola-5,7,10(19)-triene [Formula 5a; R₃=sulfonylbenzothiazolyl, R₁=t-butyl(dimethyl)silyl]

To a 3 L round bottom flask with (6RS)-SO₂-(5E,7E)-(1S,3R)-1,3-bis[t-butyl(dimethylsilyl)oxy]-22-sulfonylbenzothiazolyl-23,24-dinor-9,10-secochola-5(10),7-diene adduct 1700 mL of ethanol was added. The flask was fitted with a reflux condenser and placed in a heating bath over a magnetic stirrer. Then, 100 g of NaHCO₃ was added and continuously stirred and heated under reflux for over 3 hours (TLC, hexane-ethyl acetate 16%). The mixture was cooled, the stirrer was removed, and the solvents were removed under reduced pressure. Then, 800 mL of water and 800 mL of ethyl acetate were added. The organic phase was separated and the residue was extracted with two portions of ethyl acetate (2×250 mL). The combined organic phases were dried over anhydrous Na₂SO₄ (100 g) and filtered into a round bottom flask (3 L). Solvents were removed under reduced pressure. The residue was dissolved in toluene and injected onto a chromatography column packed with aluminium oxide (1:10) in hexane. Hexane was used to wash down the toluene on the column. The product was eluted with a 10% mixture of ethyl acetate-hexane. Solvents were removed under reduced pressure to give ca. 50 g of light yellow oily mixture of products. Then ca. 35 mL of ethyl acetate was added, and the mixture was heated near boiling point and 900 mL of methanol was added in one portion. The mixture was left at −20° C. for 24 hours. The precipitate was filtered off and washed with one portion of cold methanol (100 mL). The precipitate was dried to a constant weight on an oil vacuum pump. Obtained was ca. 24 g of product used in the next reaction without further purification. UV λₘₐₓ (EtOH) 271.0, 240.6, 207.0 nm, λₘᵢₙ 245.6, 231.0 nm; IR, v, 2951, 2928, 2883, 2856, 1636, 1554, 1472, 1324, 1252, 1147, 1083, 834, 760 cm⁻¹; ¹H-NMR (δ, ppm) 0.06 (12H, m, 2 Si(CH₃)₂), 0.56 (3H, s, 18-CH₃), 0.85 (18H, m, 2 Si—C(CH₃)₃), 1.26 (3H, d, J=6.7 Hz, 21-CH₃), 3.31 and 3.62 (2H, m, 22-CH₂), 4.22 (1H, m, 3-H), 4.53 (1H, m, 1-H), 4.97 (2H, m, 19E-H and 19Z-H), 5.79 (1H, d, J=11.5 Hz, 7-H), 6.42 (1H, d, J=11.5 Hz, 6-H), 7.62, 8.04, 8.23 (4H, m, Ar—H).

### Example 4

### Preparation of (5Z,7E)-(1S,3R)-1,3-bis[t-butyl(dimethylsilyl)oxy]-22-sulfonylbenzothiazolyl-23,24-dinor-9,10-secochola-5,7,10(19)-triene [Formula 1; R₂=sulfonylbenzothiazolyl, R₁=t-butyl(dimethyl)silyl]

(5E,7E)-(1S,3R)-1,3-Bis [t-butyl(dimethylsilyl)oxy]-22-sulfonylbenzothiazolyl-23,24-dinor-9,10-secochola-5,7,10(19)-triene (24 g) was dissolved in a mixture of toluene-methanol 5:1 (3 L), saturated with argon. Then 24 g of anthracene were added. The solution was placed in a UV irradiation apparatus and a circulating pump and a UV lamp power supply were turned on. Exposure was carried out for 3.5 hours at 18-20° C., until the disappearance of substrate (TLC, hexane-ethyl acetate 40:1). The solvents were removed under reduced pressure. Then 400 mL of hexane were added to the residue and left for 6 hours at −20° C. The mixture was filtered in vacuo through a Shott funnel, and the precipitate was washed with cold hexane (50 mL). The residue was dissolved in 450 mL of toluene and a solution of 2.4 g of maleic anhydride in 50 mL of toluene was added. The mixture was saturated with argon and stirred for 12 hours on a magnetic stirrer at room temperature. Solvents were removed under reduced pressure. The residue was dissolved in a mixture of 15 mL of toluene and 15 mL of hexane and injected onto a chromatography column filled with 350 g of silica gel 230-400 mesh. The mixtures of hexane-ethyl acetate were used as eluents: hexane 1000 ml, hexane-ethyl acetate 1% 1500 ml, hexane-ethyl acetate 2% 1000 ml, hexane-ethyl acetate 4% 2500 mL. From the combined fractions containing the product (TLC, hexane-acetate 18%) solvents were removed under reduced pressure. Obtained was ca. 21 g of product as a yellow precipitate. The precipitate was dissolved in 30 mL of ethyl acetate at temperature near boiling point and 700 mL of methanol was added in one portion. The solution was left at −20° C. for 24 hours. The precipitate was separated using a Buchner funnel, washed with cold methanol (100 mL) and dried to a constant weight on an oil vacuum pump. Obtained was 17.5 g of (5Z,7E)-(1S,3R)-1,3-bis[t-butyl(dimethylsilyl)oxy]-22-sulfonylbenzothiazolyl-23,24-dinor-9,10-secochola-5,7,10(19)-triene as a colorless fluffy powder; UV λₘₐₓ (EtOH) 268.2, 240.0, 214.4 nm, λₘᵢₙ 245.6, 231.0 nm; IR, v, 2951, 2928, 2883, 2856, 1636, 1554, 1472, 1324, 1252, 1147, 1083, 834, 760 cm⁻¹; ¹H-NMR (δ, ppm) 0.06 (12H, m, 2 Si(CH₃)₂, 0.55 (3H, s, 18-CH₃), 0.87 (18H, m, 2 Si—C(CH₃)₃), 1.26 (3H, d, J=6.6 Hz, 21-CH₃), 3.28 and 3.65 (2H, m, 22-CH₂), 4.18 (1H, m, 3-H), 4.36 (1H, m, 1-H), 4.83 (1H, m, 19Z-H), 5.16 (1H, m, 19E-H), 5.99 (1H, d, J=11.4 Hz, 7-H), 6.21 (1H, d, J=11.4 Hz, 6-H), 7.61, 8.02, 8.22 (4H, m, Ar—H).

What is claimed is:

1. A process for the preparation of a sulfonyl derivative of cholecalciferol of Formula 1, comprising the steps of
(a) reacting a compound of Formula 2b with a thiol of formula HS—R₂ to yield a compound of Formula 3b;
(b) oxidizing said compound of Formula 3b to a compound of Formula 4b;
(c) thermolysing said compound of Formula 4b to a (5E,7E)-sulfone of Formula 5b; and
(d) subjecting said (5E,7E)-sulfone of Formula 5b to a sensitized photoisomerization reaction to yield a (5Z,7E)-sulfone of Formula 1;
wherein
R₁ is a protective group;
R₂ is a heterocyclic group selected from a 2-thiazolyl, a 2-benzothiazolyl, a 1-phenyl-1H-tetrazo-5-yl, a 2-pyridyl, a 2-pyrimidynyl, a 1-isochinolinyl, a 1-methyl-2-imidazyl, and a 4-alkyl-1,2,4-triazo-3-yl; and
A is a dienophile moiety.

2. The process of claim 1, wherein A is —S(O₂)—.

3. A cholecalciferol derivative of Formula 3a, wherein
R₁ is a protective group; and
R₂ is a heterocyclic group selected from a 2-thiazolyl, a 2-benzothiazolyl, a 1-phenyl-1H-tetrazo-5-yl, a 2-pyridyl, a 2-pyrimidynyl, a 1-isochinolinyl, a 1-methyl-2-imidazyl, and a 4-alkyl-1,2,4-triazo-3-yl.

4. The cholecalciferol derivative according to claim 3, which is (6RS)-SO₂-(5E,7E)-(1S,3R)-1,3-bis[t-butyl(dimethylsilyl)oxy]-22-thiobenzothiazolyl-23,24-dinor-9,10-secochola-5(10),7-diene.

5. A cholecalciferol derivative of Formula 4a, wherein
R₁ is a protective group; and
R₂ is a heterocyclic group selected from a 2-thiazolyl, a 2-benzothiazolyl, a 1-phenyl-1H-tetrazo-5-yl, a 2-pyridyl, a 2-pyrimidynyl, a 1-isochinolinyl, a 1-methyl-2-imidazyl, and a 4-alkyl-1,2,4-triazo-3-yl.

6. The cholecalciferol derivative according to claim 5, which is (6RS)-SO₂-(5E,7E)-(1S,3R)-1,3-bis[t-butyl(dimethylsilyl)oxy]-22-sulfonylbenzothiazolyl-23,24-dinor-9,10-secochola-5(10),7-diene.

7. The process of claim 1 wherein said protective group is a t-butyl(dimethyl)silyl.

8. The process of claim 1 wherein the reaction in step (a) is carried out under Mitsunobu reaction conditions in the presence of triphenylphosphine and diisopropyl azodicarboxylate in methylene chloride at lower temperatures.

9. The process of claim 1 wherein the reaction in step (b) is carried out with ammonium molybdate tetrahydrate-hydrogen peroxide in ethanol at elevated temperatures.

10. The process of claim 9 wherein the reaction in step (b) is carried out at a temperature between 50 and 80° C.

11. The process of claim 1 wherein the reaction in step (c) is carried out with sodium bicarbonate in an alcohol under reflux.

12. The process of claim 11 wherein said alcohol is selected from methanol, ethanol, butanol, and ethylene glycol.

13. The process of claim 1 wherein the reaction in step (d) is carried out with UV radiation in the presence of anthracene.

14. The cholecalciferol derivative of claim 3, wherein R₂ is a t-butyl(dimethyl)silyl.

15. A process for converting a sulfide derivative of cholecalciferol of Formula 3 having a triene system to a sulfone derivative of cholecalciferol of Formula 1 by oxidation, wherein
for the purpose of said oxidation, said triene system is protected as a Diels-Alder adduct with a dienophile, and after said oxidation a resultant Diels-Alder adduct is deprotected in a retro-Diels-Alder reaction,
R₁ is a protective group; and
R₂ is a heterocyclic group selected from a 2-thiazolyl, a 2-benzothiazolyl, a 1-phenyl-1H-tetrazo-5-yl, a 2-pyridyl, a 2-pyrimidynyl, a 1-isochinolinyl, a 1-methyl-2-imidazyl, and a 4-alkyl-1,2,4-triazo-3-yl.

16. The process of claim 15 wherein said sulfide derivative of cholecalciferol of Formula 3 has a (5Z,7E)-configuration.

17. The process of claim 15 wherein said sulfone derivative of cholecalciferol of Formula 1 has a (5Z,7E)-configuration.

18. The process of claim 15 wherein after said oxidation said resultant Diels-Alder adduct is deprotected by thermolysis.

19. The process of claim 15 wherein R₁ is a t-butyl(dimethyl)silyl.

20. The process of claim 15 wherein R₂ is a 2-benzothiazolyl.

21. The process of claim 15 wherein said dienophile is sulfur dioxide.

22. A process for the preparation of a sulfonyl derivative of cholecalciferol of Formula 1, comprising the steps of
(a) reacting a compound of Formula 2a with a thiol of formula HS—R₂ to yield a compound of Formula 3a;
(b) oxidizing said compound of Formula 3a to a compound of Formula 4a;
(c) thermolysing said compound of Formula 4a to a (5E,7E)-sulfone of Formula 5a; and
(d) subjecting said (5E,7E)-sulfone of Formula 5a to a sensitized photoisomerization reaction to yield a (5Z,7E)-sulfone of Formula 1;
wherein
R₁ is a protective group; and
R₂ is a heterocyclic group selected from a 2-thiazolyl, a 2-benzothiazolyl, a 1-phenyl-1H-tetrazo-5-yl, a 2-pyridyl, a 2-pyrimidynyl, a 1-isochinolinyl, a 1-methyl-2-imidazyl, and a 4-alkyl-1,2,4-triazo-3-yl.

23. The process of claim 22 wherein R₁ is a t-butyl(dimethyl)silyl.
